(19) United States
(12) Patent Application Publication
(10) Pub. No.: US 2025/0177034 A1
(43) Pub. Date: Jun. 5, 2025

(54) HIGH FREQUENCY VASCULAR CLOSURE DEVICE

(71) Applicant: TERUMO MEDICAL CORPORATION, Somerset, NJ (US)

(72) Inventor: Tomoji Maruyama, Bear, DE (US)

(21) Appl. No.: 19/049,646

(22) Filed: Feb. 10, 2025

Related U.S. Application Data
(63) Continuation of application No. 16/595,820, filed on Oct. 8, 2019, now Pat. No. 12,256,977.

(51) Int. Cl. A61B 18/14; A61B 17/00; A61B 17/28; A61B 18/00; A61B 18/12

(52) U.S. Cl. CPC A61B 18/1445; A61B 17/0057; A61B 17/2833; A61B 2017/00654; A61B 2017/00778; A61B 2018/00077; A61B 2018/00404; A61B 2018/126; A61B 2018/1475

(57) ABSTRACT

A vascular closure device seals a vascular access site upon completion of a procedure wherein a blood vessel has been punctured to accommodate a guidewire, an introducer sheath, and other intravascular devices. The device uses compression and thermal heating of an area around the puncture to obtain immediate hemostasis. Electrothermal bipolar vessel sealing is employed, wherein a forceps grasps and compresses tissue from opposite sides of the puncture and then applies a high-frequency alternating electric current to cause the vessel wall between the forceps to denature and seal.

### CROSS-REFERENCE TO RELATED APPLICATION

This application is a continuation of U.S. application Ser. No. 16/595,820, filed on Oct. 8, 2019. The disclosure of the prior application is hereby incorporated by reference in its entirety.

### BACKGROUND

The present disclosure relates in general to vascular closure devices, and, more specifically, to sealing a vascular access site upon completion of a procedure wherein a blood vessel has been punctured to accommodate a guidewire, an introducer sheath, and other intravascular devices.

Certain medical procedures utilize the introduction of instruments and devices into a body via a blood vessel (e.g., a femoral artery or a radial artery). Examples include coronary angiography and angioplasty. The procedures involve the creation of a puncture in the blood vessel by inserting a small catheter/needle to create the puncture, followed by insertion of a plastic introducer sheath which maintains the opening during insertion of a guidewire and other devices to perform a desired diagnostic or therapeutic intervention. Upon completion of the intervention, the devices are removed and it is necessary to close the puncture site to achieve hemostasis in an effective and efficient manner.

Conventional techniques used to stop the bleeding from a puncture include external compression (e.g., manual compression for at least 10 to 15 minutes), internal compression (e.g., using an inflatable balloon), suturing, and insertion of a biodegradable plug. It would be desirable to improve patient outcomes by reducing the time required for obtaining hemostasis, alleviating any requirement for immobilizing the patient for prolonged periods, and avoiding the need to implant or leave behind any foreign substances.

### SUMMARY

The present disclosure utilizes compression and thermal heating of an area around the puncture to obtain immediate sealing of the blood vessel. In one embodiment, electrothermal bipolar vessel sealing is employed wherein a clamp or forceps grasps and compresses tissue from opposite sides of the puncture and then applies a high-frequency alternating electric current to cause the vessel wall between the forceps to denature and seal. Sealing of vessels by thermal heating has generally been used as a technique for closing the end of a severed vessel, but has not been successfully implemented for closing punctures in a vessel wall.

In one aspect of the present disclosure, a closure device is provided for sealing a puncture in a blood vessel. The device has a housing with a distal shaft and a proximal casing oriented along a longitudinal axis, wherein the housing defines a longitudinal passage to receive a guidewire. The device has an inner housing disposed in an internal chamber of the casing, wherein the inner housing is slidable along the longitudinal axis between a distal position and a proximal position. The inner housing is biased into the distal position. A forceps is mounted at a distal end of the distal shaft comprising first and second jaws, wherein the first and second jaws are electrically conductive and are electrically insulated from each other. The first and second jaws are pivotable between an open position and a closed position for grasping the blood vessel around the puncture to be sealed. First and second electrical conductors have their distal ends connected to the first and second jaws, respectively. Their proximal ends are configured to receive a bipolar electrical current (e.g., from a high-frequency generator). A positioning wire is retained by the distal shaft. The positioning wire is selectably extendable from a retracted position contained in the distal shaft to an extended position with a distal end protruding from the distal shaft beyond the forceps. The protruding distal end is comprised of a shape memory material that reconfigures from a substantially straight shape when stored in the retracted position into a lateral profile extending transverse to the longitudinal axis when in the extended position. A positioning lever is mounted to the inner housing and coupled to the positioning wire. The positioning lever is movable between a first position that places the positioning wire in the retracted position and a second position that places the positioning wire in the extended position, wherein the positioning lever is biased to the first position. A forceps lever is mounted to the inner housing and coupled to the first and second jaws. The forceps lever is movable between a first position that places the jaws in the closed position and a second position that places the jaws in the open position, and the forceps lever is biased to the first position.

The device is positionable along the guidewire to approach the puncture. The positioning lever is movable from the first position to the second position to place the positioning wire into the lateral profile inside the blood vessel. The distal end of the positioning wire centers the puncture with respect to the forceps when the device is partially withdrawn along the guidewire. The inner housing has a first locking feature to capture the positioning lever in the second position. The forceps lever is movable from the first position to the second position to pivot the jaws into the open position spanning the puncture. The inner housing has a second locking feature to capture the forceps lever in the second position. The inner housing is movable from the distal position to the proximal position to cause the positioning wire to retract into the distal shaft and the jaws to pivot to the closed position grasping the blood vessel across the puncture so that the puncture can be closed by activating the bipolar electrical current.

### BRIEF DESCRIPTION OF THE DRAWINGS

FIG. 1 is a diagram showing an interventional system introduced subcutaneously into a patient's blood vessel.

FIG. 2 is a side view showing an introducer sheath and blood vessel in greater detail.

FIG. 3 is a side view of a closure device of the present disclosure interconnected with a bipolar power supply.

FIG. 4 is a side, cross-sectional view of an outer housing of the device of FIG. 3.

FIG. 5 is a side view of the device of FIG. 3 with the outer housing shown in cross section to reveal an inner housing and other components.

FIG. 6 is a side view of a proximal portion of the device of FIG. 3 with the outer housing and inner housing shown in cross section.

FIG. 7 is a side view of a distal portion of the device of FIG. 3 shown in partial cross section.

FIG. 8 is a perspective view of a jaw of the forceps of the device of FIG. 3.

FIG. 9 is a perspective view showing a portion of the proximal portion of the device of FIG. 3 with a guide slot.

FIG. 10 is a side view of the proximal portion shown in FIG. 6 with the positioning lever moved to a second position which extends a positioning wire.

FIG. 11 shows an inner tube and a distal end of the positioning wire in a retracted position.

FIG. 12 shows an inner tube and a distal end of the positioning wire in an extended position.

FIG. 13 shows the extended positioning wire of FIG. 12 as seen from a vantage point transverse to the vantage point of FIG. 12.

FIG. 14 is a partial cross-sectional view showing the positioning wire inside the blood vessel, wherein the cross section is taken along an axial direction of the blood vessel.

FIG. 15 is a partial cross-sectional view showing the positioning wire inside the blood vessel, wherein the cross section is taken perpendicular to the axial direction of the blood vessel.

FIG. 16 is a side view of the proximal portion shown in FIG. 6 with the forceps lever moved to a second position which pivots the jaws of the forceps to an open position.

FIG. 17 shows the jaws of the forceps pivoted to the open position, ready to receive a vessel wall for compression and thermal heating.

FIG. 18 is a partial cross-section of the proximal portion of the device of FIG. 3 with the outer housing shown in cross section to reveal the inner housing withdrawn to a proximal position.

FIG. 19 is a flowchart showing one embodiment of a method of the present disclosure.

### DETAILED DESCRIPTION

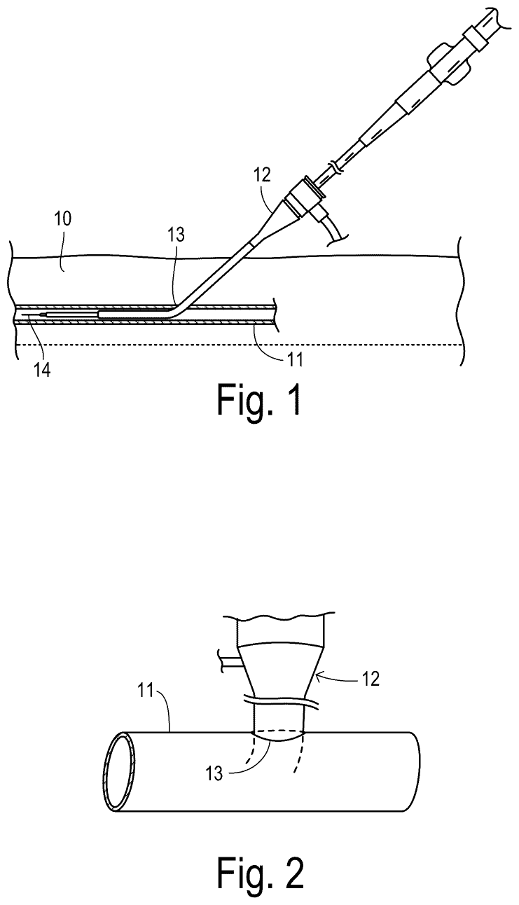

In FIGS. 1 and 2, a body 10 of a patient contains a vessel blood vessel 11 which is punctured by an introducer sheath 12 at a puncture site 13. A guidewire 14 passes through sheath 12 and vessel 11 for guiding the introduction of various devices. Upon completion of an intervention, the devices are removed from guidewire 14 so that only guidewire 14 and sheath 12 remain. Thereafter, the tool of the present disclosure may be introduced over guidewire 14 to approach the puncture site 13 in order to close the blood vessel 11 as described below.

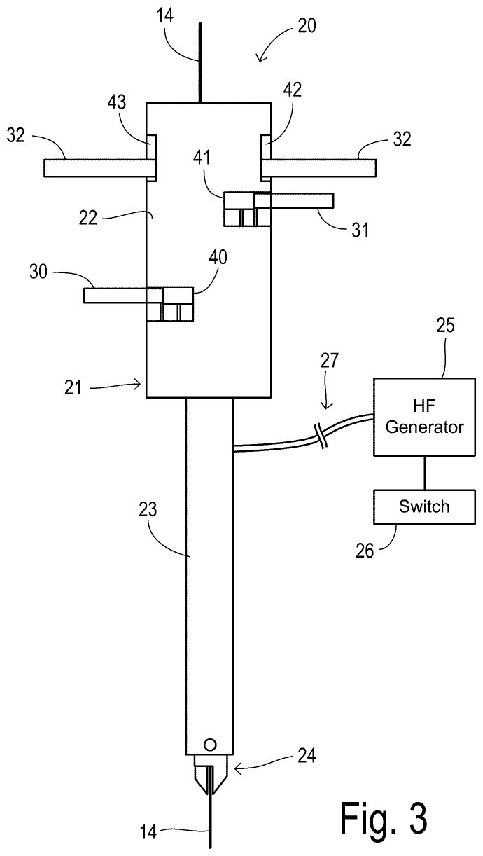

FIG. 3 shows a closure device 20 with a longitudinal passage installed on guidewire 14. Device 20 has a housing 21 with a proximal casing 22 and a distal shaft 23 arranged along a longitudinal axis. A forceps 24 is mounted at a distal end of shaft 23 and is arranged to receive a bipolar alternating electrical current from a high-frequency (HF) generator 25 under control of a switch 26 via electrical conductors 27. Switch 26 may include a manual pushbutton or a foot pedal, for example. Generator 25 can be a type that is commercially available for performing electrothermal bipolar vessel heating. For example, generator 25 may monitor impedance during heating in order to optimize vessel sealing without charring.

Housing 21 accommodates other mechanisms described below in connection with obtaining a proper placement of device 20 and the manipulation of forceps 24. These include a first (positioning) lever 30, a second (forceps) lever 31, and a third (inner housing) lever 32, each of which is movable longitudinally to operate various portions of device 20 as explained below.

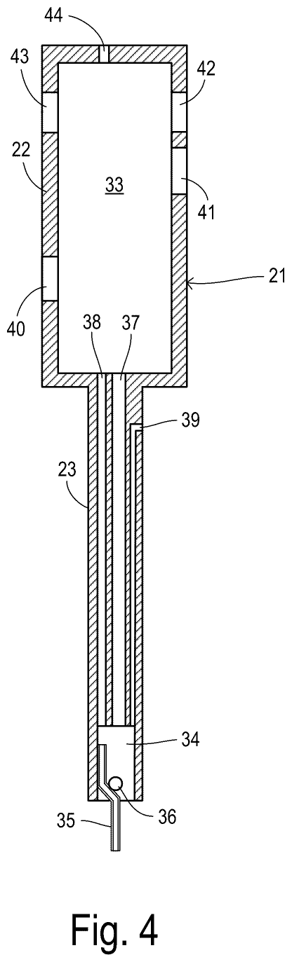

FIG. 4 shows housing 21 in cross-section to reveal an internal chamber 33 within proximal casing 22. Distal shaft 23 defines an end chamber 34 to receive forceps 24. An inner tube 35 is disposed in chamber 34 and has a distal end which is aligned with the central axis of chamber 34. An aperture 36 receives a pivot pin of forceps 24. Distal shaft 23 includes one or more passages such as passages 37, 38, and 39 to accommodate components such as an actuation pin for the forceps, a positioning wire, and electrical conductors. Instead of forming passage 38 directly within the body of shaft 23, inner tube 35 could extend the full length of distal shaft 23 (i.e., within a cylindrical bore of shaft 23). In addition, passage 39 could be combined with passage 37 (e.g., shaft 23 could provide an outer tube for carrying the actuation pin, the conductor wires, and inner tube 35, while inner tube 35 conveyed guidewire 14 and the positioning wire).

Proximal casing 22 includes side openings 40 and 41 for receiving levers 30 and 31, respectively, and side openings 42 and 43 for receiving lever 32. The levers include radial handles projecting through the openings from respective internal sliding bodies. An opening 44 is provided for receiving guide wire 14.

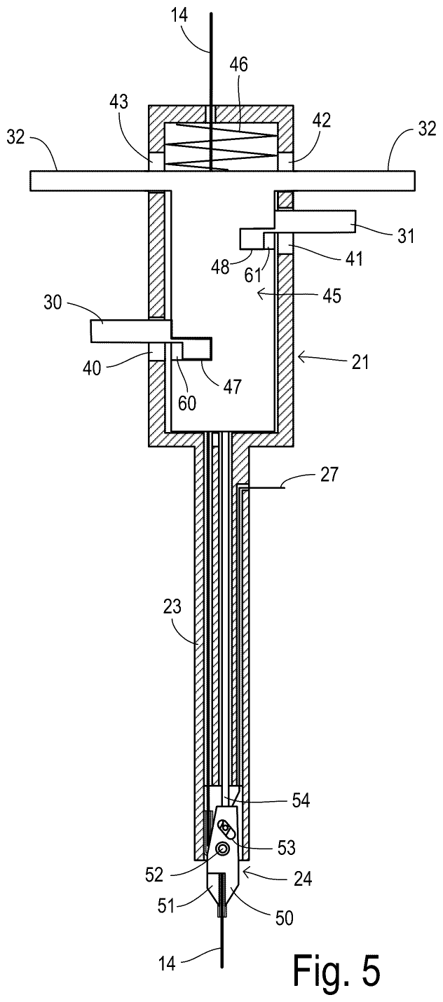

FIG. 5 shows an inner housing 45 installed in internal chamber 33, wherein inner housing 45 is slidable along the longitudinal axis between a distal position as shown in FIG. 5 and a proximal position (see FIG. 18). A spring 46 biases inner housing 45 into the distal position. In the distal position, lever 32 extends from inner housing 45 and bears against distal edges of openings 42 and 43. In the proximal position, lever 32 bears against proximal edges of openings 42 and 43. Also when inner housing 45 is in the distal position, levers 30 and 31 are located at proximal edges of openings 30 and 31. Levers 30 and 31 are longitudinally movable within the inner housing 45, and can be moved in a distal direction and then rotated so that levers 30 and 31 enter locking sections 47 and 48 of corresponding openings in inner housing 45 as explained below.

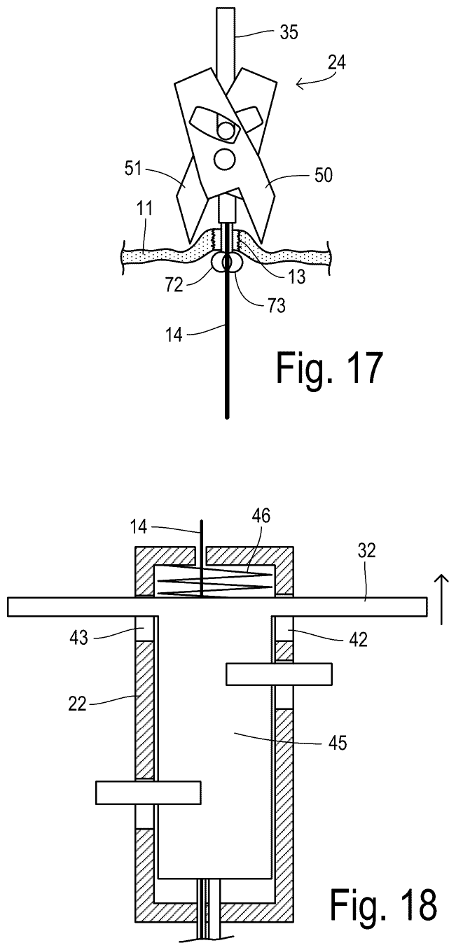

As shown in FIG. 5, forceps 24 includes jaws 50 and 51 which are installed on a pivot pin 52 to be pivotable between a closed position as shown in FIG. 5 and an open position (see FIG. 17). Jaws 50 and 51 are electrically conductive and are connected with electrical conductors 27 in a known manner (e.g., soldering) in order to facilitate application of bipolar electrical current by jaws 50 and 51 to tissue of a vessel wall that is compressed between them. Jaws 50 and 51 are electrically insulated from each other and from the surrounding structures such as distal shaft 23 and guidewire 14 (e.g., using insulating coatings on selected surfaces). Jaw 50 includes a slanted slot 53 for receiving an end of an actuator pin 54 which is operated by lever 31, whereby longitudinal movement of pin 54 causes pivoting motion of jaw 50. Jaw 51 has a similar slot slanted in an opposite direction in order to pivot in opposition to jaw 50 (see FIG. 7).

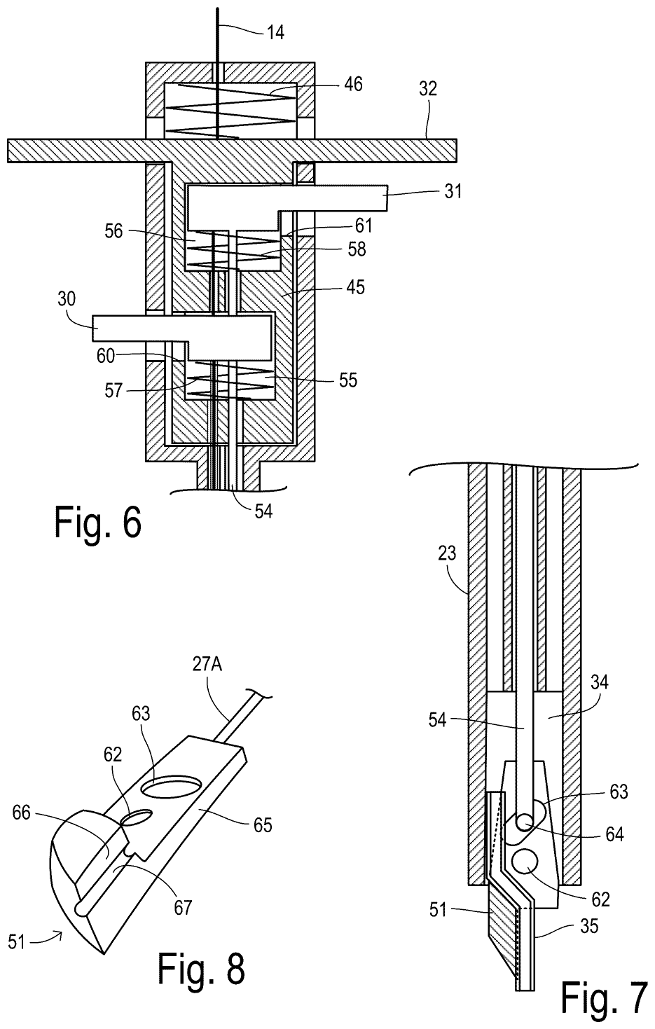

FIG. 6 shows inner housing 45 in cross-section to reveal chambers 55 and 56 which receive levers 30 and 31, respectively, in a longitudinally slidable manner. Levers 30 and 31 are biased into first (proximal) positions by springs 57 and 58. Inner housing 45 has guide slots 60 and 61 for accommodating levers 30 and 31. Guide slots 60 and 61 include locking sections 47 and 48 so that levers 30 and 31 may be manually moved to second (distal) positions against bias springs 57 and 58 in a longitudinal direction and then rotated so that levers 30 and 31 enter locking sections 47 and 48, thereby locking them at the second positions. The levers can be unlocked and returned to the biased positions by counter-rotation to exit the locking sections. Lever 30 is affixed to a positioning wire described in more detail below.

Actuator pin 54 extends from lever 31 in a continuous manner by passing through a central aperture in lever 30 (not shown). Guidewire 14 may pass along an edge of chambers 55 and 56 so as not to interfere with operation of levers 30 and 31.

FIG. 7 shows the distal end of distal shaft 23 in greater detail (jaw 50 is removed). Jaw 51 includes an aperture 62 for receiving pivot pin 52 and a slanted slot 63 receiving an end of actuator pin 54. The distal end 64 of actuator pin 54 may have a “T” shape with respective crossmembers entering slanted slots 53 and 63.

Jaw 51 is shown in greater detail in FIG. 8. A main body 65 is electrically connected to a first electrical conductor 27A at a proximal end. A second conductor 27B connects to jaw 50 (not shown). A compression surface 66 is provided at the distal, inside-facing of jaw 51 for compressing tissue (i.e., the vessel wall) with a corresponding compression surface of jaw 50. One or both of the jaws further includes a groove 67 extending longitudinally for accommodating the passage of guidewire 14 and the positioning the wire to be described below.

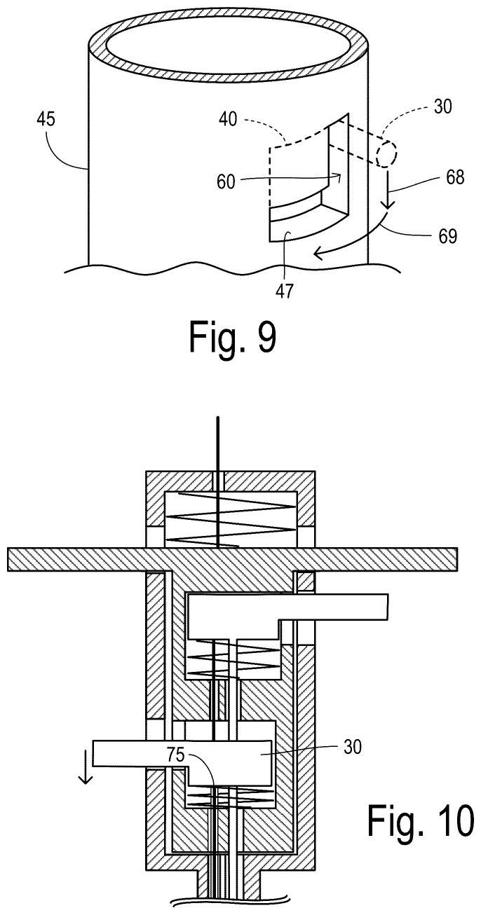

FIG. 9 shows a perspective view of a portion of inner housing 45 including opening 60 which acts as a guide slot for lever 30. Guide slot 60 has a longitudinal arm adapted to allow longitudinal movement of lever 30 and a circumferential arm which forms locking section 47. The radial handle portion of lever 30 projects radially through guide slot 60 and is movable in the longitudinal arm along a direction 68 to move lever 30 and its actuation pin from the first (proximal) position to the second (distal) position. Then lever 30 can be rotated along the circumferential arm in the direction of arrow 69 to lock lever 30 in the second position. The openings in outer housing 21 span an area to accommodate all possible movements of the levers within inner housing 45 (e.g., square profiles that expose the guide slots of inner housing 45). FIG. 9 has a dashed line to show a portion of a footprint corresponding to the position of opening 40 of outer housing 21 when projected onto inner housing 45.

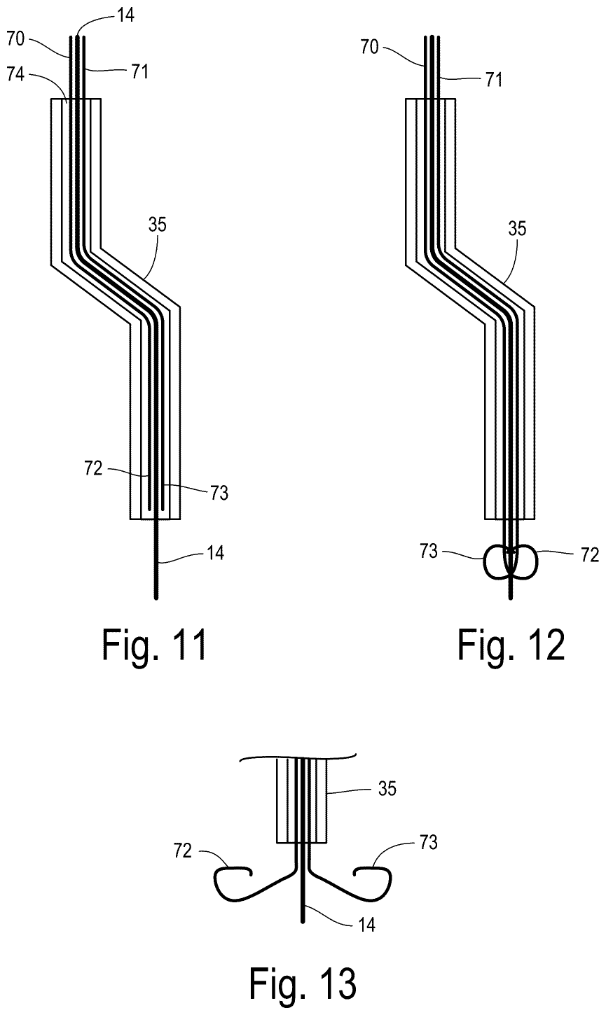

FIGS. 10-13 illustrate the positioning wire of the present disclosure which serves to align tool 20 and puncture 13 under control of lever 30. The positioning wire may include at least two wire members 70 and 71, each of which are fixed at their proximal ends to a bottom edge 75 of positioning lever 30. Therefore, positioning wires 70 and 71 are selectively extendable by moving positioning lever 30 to the second (distal) position as shown in FIG. 10. FIGS. 11 and 12 show positioning wires 70 and 71 passing through a lumen 74 in inner tube 35. FIG. 11 shows a retracted position with distal ends 72 and 73 of positioning wires 70 and 71, respectively, stored inside lumen 74. In this position, distal ends 72 and 73 are kept substantially straight by inner tube 35. At least at the distal ends 72 and 73, positioning wires 70 and 71 are comprised of a shape memory material such as nitinol. The shape memory material is constructed to provide an unrestrained shape that follows a lateral profile wherein positioning wires 70 and 71 extend transversely to the longitudinal axis of tool 20. In particular, distal ends 72 and 73 deploy generally at a right angle with respect to the axial direction of inner tube 35. The lateral profile of the memory shape is adapted to span a size larger than the diameter of puncture 13. Furthermore, the lateral profile is generally oblong, having a maximum length perpendicular to the pivot plane of the forceps jaws and a minimum length parallel to the pivot plane. In one embodiment, each positioning wire distal end 72 and 73 turns sideways after extending outward from inner tube 35 and follows a quasi-spiral shape which proceeds radially outward and then bends back in to extend radially inward. This provides a bearing surface that can engage an inner surface of the wall of the blood vessel. FIGS. 12 and 13 show the extended position with distal ends 72 and 73 protruding from inner tube 35. In FIG. 12, the minimum side-to-side length of the lateral profile is seen (which coincides with the pivot plane). FIG. 13 is a view which is rotated by 90° to show the maximum side-to-side length of the lateral profile which occurs transverse to the pivot plane. Thus, the portions of positioning wire distal ends 72 and 73 that make significant contact with the vessel wall are separated from (i.e., rotated by 90° from) the contact points for the forceps jaws, which encourages the edges of the vessel wall around the puncture to bulge upward for grasping by the jaws.

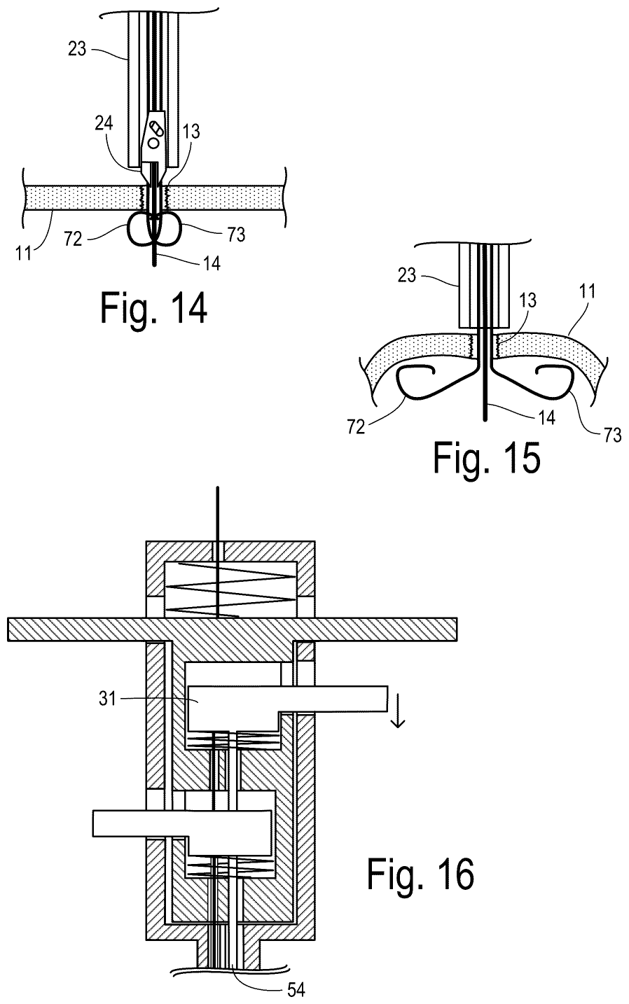

In order to align device 20 for bipolar sealing, it is advanced on guidewire 14 until its distal end approaches the puncture. Then, positioning wires 70 and 71 are extended out from inner tube 35 so that distal ends 72 and 73 deploy transversely according to the lateral profile while inside blood vessel 11 as shown in FIG. 14. FIG. 14 depicts a cross-section along the axial length of blood vessel 11. In this orientation, the pivot plane of forceps 24 is arranged lengthwise along vessel 11 so that grasping of the vessel walls on opposite sides of puncture 13 pull the vessel walls longitudinally when the forceps jaws are closed. The orientation is obtained when the minimum length of the lateral profile defined by distal ends 72 and 73 is arranged lengthwise in the vessel 11. FIG. 15 shows a radial cross-section of blood vessel 11 showing that the maximum length of the lateral profile is arranged perpendicular to the pivot plane (i.e., transverse to the blood flow direction) when in the orientation. Furthermore, FIGS. 14 and 15 show the condition wherein device 20 has been slightly withdrawn along guidewire 14 (after extending the positioning wires) so that the deployed portions of positioning wires 72 and 73 have been pulled into contact with the vessel wall (simultaneously pulling puncture 13 into central alignment). After achieving this condition, forceps lever 31 can be deployed in a distal direction as shown in FIG. 16 and then locked in the second (distal) position. Consequently, actuation pin 54 moves in a distal direction causing jaws 50 and 51 of forceps 24 to open as depicted in FIG. 17. The interaction between forceps 24 and positioning wire distal ends 72 and 73 results in portions of the vessel wall around puncture 13 squeezing up between the opposing compression surfaces of jaws 50 and 51. In this state, inner housing 45 will then be retracted from its distal position to the proximal position as shown in FIG. 18 by pulling upward (proximally) on lever 32. Lever 32 moves upward in openings 42 and 43 while bias spring 46 is compressed. The retracting movement causes positioning wires 70 and 71 to retract into inner tube 35 and forceps jaws 50 and 51 to close. In that position, the HF generator can be activated and the puncture is closed by thermal heating. Guidewire 14 may first be withdrawn prior to activating the thermal heating.

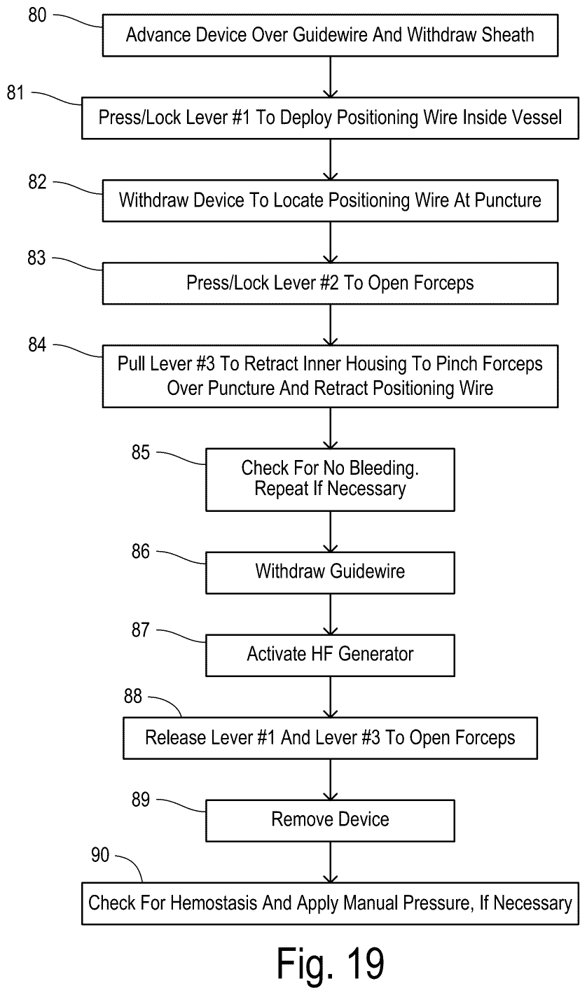

A method of the present disclosure is summarized in FIG. 19. Instruments and devices related to an intervention would first be removed from the guidewire so that only the guidewire and the introduced sheath remain. In step 80, the device is advanced over the guidewire so that the forceps and inner tube approach the puncture. The introducer sheath is withdrawn, and it may remain over the distal shaft of the device where it will not interfere with the closing of the puncture. In step 81, the first (positioning) lever for the positioning wire is advanced (e.g., pressed) in a distal direction and then locked in order to deploy the positioning wire inside the blood vessel (as shown in FIG. 10). Consequently, the expanded shape of the distal ends of the positioning wire is deployed on the inside of the blood vessel. In step 82, the device is partially or slightly withdrawn in order to locate the positioning wire at the puncture (as shown in FIGS. 14 and 15) in order to accurately position the puncture site, align the location of the blood vessel with the area to be grasped by the forceps, and to provide a reaction surface for the forceps to push against as they grasp the vessel walls. The radial handles of the inner housing lever handles extend parallel (i.e., radially coplanar) to the pivot plane in order to assist the user in orienting the forceps along the lengthwise direction of the vessel (which simultaneously orients the maximum length of the deployed positioning wires in a transverse direction).

With the positioning wires and puncture properly oriented, the forceps lever is advanced (as shown in FIG. 16) by pressing in the distal direction and locking the forceps lever so that the forceps jaws are in an open position (as shown in FIG. 17). In step 84, the radial handles of the inner housing lever are pulled in a proximal direction (as shown in FIG. 18) so that the inner housing retracts to the proximal position, resulting in the forceps jaws pinching together opposing sides of the vessel wall from opposite sides of the puncture, and simultaneously retracting the positioning wires. If successful, the pinched vessel walls will temporarily close the puncture and bleeding will cease. In step 85, the user may check for any bleeding. If bleeding is present, then each of the levers may be restored to their biased positions and steps 82-85 may be repeated until the bleeding has been sufficiently stopped. Then the guidewire is withdrawn in step 86 and the HF generator is activated in step 87.

The HF generator utilizes known techniques in order to apply an appropriate bipolar current magnitude and duration that successfully denatures and seals the vessel around the puncture. Then the inner housing lever and the positioning wire lever are released in step 88, resulting in opening of the forceps jaws while the positioning wire remains in the retracted position. Then the device can be removed in step 89. In step 90, a further check may be performed to determine whether hemostasis has been achieved. If there is any residual bleeding than manual pressure can be applied until hemostasis is achieved.

What is claimed is:

1. A device for closing a puncture in a blood vessel, comprising:
a housing comprising a longitudinal axis, a distal portion, and a proximal portion;
an inner housing slidably disposed within the proximal portion of the housing;
a forceps mounted at a distal end of the distal portion and comprising a first jaw pivotably coupled to a second jaw, the forceps is movable between an open position and a closed position;
an electrical conductor electrically coupled to the forceps, the electrical conductor is configured to transmit an electrical current to one or more of the first jaw and the second jaw;
a positioning wire extending through the distal portion of the housing and selectably movable between (i) a retracted position in which a distal end of the positioning wire is proximal to a distal end of the distal portion, and (ii) an extended position in which the distal end of the positioning wire is distal to the distal end of the distal portion;
a positioning lever mounted inside the inner housing and coupled to the positioning wire, wherein the positioning lever is movable between (a) a first position that places the positioning wire in the retracted position, and (b) a second position that places the positioning wire in the extended position; and
a forceps lever coupled to the inner housing and coupled to the forceps, wherein the forceps lever is movable between (I) a first position that places the forceps in the closed position, and (II) a second position that places the forceps in the open position.

2. The device of claim 1, wherein the inner housing is slidable along the longitudinal axis between a distal position and a proximal position,
wherein the device is configured such that when the inner housing moves from the distal position to the proximal position, the positioning wire retracts into the distal portion of the housing and the forceps moves to the closed position.

3. The device of claim 2, comprising:
an inner housing lever extending radially outward from an exterior of the proximal portion of the housing; and
an inner housing spring disposed in the inner housing and coupled to the inner housing lever,
wherein the inner housing spring is movable between a compressed configuration in which the inner housing is in the proximal position and an expanded configuration in which the inner housing is in the distal position.

4. The device of claim 1, comprising a positioning lever spring coupled to the positioning lever and disposed inside the inner housing,
wherein the positioning lever spring is movable between (i) a compressed configuration in which the positioning lever is in the second position, and (ii) an expanded configuration in which the positioning lever is in the first position.

5. The device of claim 1, comprising a forceps lever spring coupled to the forceps lever and disposed inside the inner housing,
wherein the forceps lever spring is movable between (i) a compressed configuration in which the forceps lever is in the second position, and (ii) an expanded configuration in which the forceps lever is in the first position.

6. The device of claim 1, wherein the forceps lever is biased to the first position, the positioning lever is biased to the first position, and the inner housing is biased into a distal position relative to the housing.

7. The device of claim 1, wherein the forceps lever is mounted inside the inner housing.

8. The device of claim 1, wherein the device is configured such that when the positioning lever is in the second position, the positioning lever is rotatable relative to the inner housing.

9. The device of claim 8, wherein the inner housing comprises a guide slot configured to receive a portion of the positioning lever, wherein the guide slot comprises:
a longitudinal section configured to allow longitudinal movement of the positioning lever; and
a circumferential section configured to allow rotational movement of the positioning lever.

10. The device of claim 1, wherein the device is configured such that when the forceps lever is in the second position, the forceps lever is rotatable relative to the inner housing.

11. The device of claim 10, wherein the inner housing comprises a guide slot configured to receive a portion of the forceps lever, wherein the guide slot comprises:
a longitudinal section configured to allow longitudinal movement of the forceps lever; and
a circumferential section configured to allow rotational movement of the forceps lever.

12. The device of claim 1, wherein the device is configured such that each of the first and second jaws extends across and bisects the longitudinal axis when the forceps is in the open position.

13. A device for closing a puncture in a blood vessel, comprising:
a housing comprising a longitudinal axis, a distal portion, and a proximal portion;
a forceps mounted at a distal end of the distal portion and comprising a first jaw pivotably coupled to a second jaw, wherein the forceps is movable between an open position and a closed position;
an electrical conductor electrically coupled to the forceps, wherein the electrical conductor is configured to transmit an electrical current to one or more of the first jaw and the second jaw;
a positioning wire extending through the distal portion of the housing and selectably movable between (i) a retracted position in which a distal end of the positioning wire is proximal to a distal end of the distal portion, and (ii) an extended position in which the distal end of the positioning wire is distal to the distal end of the distal portion; and
an actuation assembly comprising:
an inner housing slidably disposed within the proximal portion of the housing;
a positioning lever mounted inside the inner housing and coupled to the positioning wire, wherein the positioning lever is movable between (a) a first position that places the positioning wire in the retracted position, and (b) a second position that places the positioning wire in the extended position; and
a forceps lever coupled to the inner housing and coupled to the forceps, wherein the forceps lever is movable between (I) a first position that places the forceps in the closed position, and (II) a second position that places the forceps in the open position.

14. The device of claim 13, wherein the distal end of the positioning wire comprises a shape memory material that reconfigures between (i) a substantially straight shape when stored in the retracted position and (ii) a lateral profile extending transverse to the longitudinal axis when in the extended position, and
wherein the device is configured such that when the positioning wire is in the extended position, the distal end of the positioning wire extends distally beyond the forceps.

15. The device of claim 14, wherein the first and second jaws are configured to be pivoted in a pivot plane, and
wherein the lateral profile of the positioning wire in the extended position has a maximum length perpendicular to the pivot plane and a minimum length parallel to the pivot plane.

16. A method of using a closure device, the method comprising:
advancing the closure device along a guidewire to approach a puncture in a blood vessel, wherein the closure device comprises a housing comprising a longitudinal axis, a distal portion, and a proximal portion, an inner housing slidably disposed within the proximal portion of the housing, a forceps mounted at a distal end of the distal portion, an electrical conductor electrically coupled to the forceps, and a positioning wire extending through the distal portion of the housing;
advancing a positioning lever of the closure device that is coupled to the positioning wire and that is mounted inside the inner housing from (i) a first position in which the positioning wire is in a retracted position to (ii) a second position in which the positioning wire is in an extended position;
locking the positioning lever in the second position to place a distal end of the positioning wire inside the blood vessel, wherein the distal end of the positioning wire comprises a lateral profile in which the distal end extends transverse to the longitudinal axis;
partially withdrawing the closure device along the guidewire so that the distal end of the positioning wire centers the puncture with respect to the forceps, wherein the forceps comprises first and second jaws electrically coupled to first and second conductors, respectively;
advancing a forceps lever of the closure device that is coupled to the forceps and that is mounted to the inner housing from (a) a first position in which the first and second jaws are in a closed position to (b) a second position in which the first and second jaws are in an open position;
locking the forceps lever in the second position to allow the first and second jaws to span the puncture when the forceps is in the open position;
grasping the blood vessel across the puncture by moving the forceps from the open position to the closed position; and
activating an electrical current through the first and second conductors to thermally heat the blood vessel grasped between the first and second jaws.

17. The method of claim 16, comprising moving the inner housing relative to the housing from a distal position to a proximal position to (i) retract the positioning wire into the distal portion of the housing and (ii) pivot the first and second jaws to move the forceps into the closed position.

18. The method of claim 17, comprising:
unlocking and moving the positioning lever from the second position to the first position; and
moving the inner housing from the proximal position to the distal position to allow the first and second jaws to pivot to the open position.

19. The method of claim 16, wherein advancing the positioning lever comprises longitudinally advancing the positioning lever in a distal direction, and
wherein locking the positioning lever in the second position comprises rotating the positioning lever relative to the longitudinal axis to lock the positioning lever in the second position.

20. The method of claim 16, wherein advancing the forceps lever comprises longitudinally advancing the forceps lever in a distal direction, and
wherein locking the forceps lever in the second position comprises rotating the forceps lever relative to the longitudinal axis to lock the forceps lever in the second position.
